(19) United States
(12) Patent Application Publication
(10) Pub. No.: US 2013/0060031 A1
(43) Pub. Date: Mar. 7, 2013

(54) PROCESS FOR THE PREPARATION OF HIGHLY PURE AMBRISENTAN

(71) Applicants: Amala Kompella, Hyderabad (IN); Kali Satya Bhujanga Ra Adibhatla, Hyderabad (IN); Veera Swamy Balina, Hyderabad (IN); Srinivasu Kasa, Hyderabad (IN); Venkaiah Chowdary Nannapaneni, Hyderabad (IN)

(72) Inventors: Amala Kompella, Hyderabad (IN); Kali Satya Bhujanga Ra Adibhatla, Hyderabad (IN); Veera Swamy Balina, Hyderabad (IN); Srinivasu Kasa, Hyderabad (IN); Venkaiah Chowdary Nannapaneni, Hyderabad (IN)

(73) Assignee: Natco Pharma Limited, Hyderabad (IN)

(21) Appl. No.: 13/635,299

(22) PCT Filed: Mar. 15, 2010
(86) PCT No.: PCT/IN2010/000153

§ 371 Date: Nov. 20, 2012

(51) Int. Cl. C07D 239/34

(52) U.S. Cl. 544/318

(57) ABSTRACT

The present invention relates to an improved and novel process for the preparation of highly pure (>99.8%) (+)-2(S)-(4,6-dimethylpyrimidin-2-yloxy)-3-methoxy-3,3-diphenylpropionic acid (Ambrisentan) of formula (I).

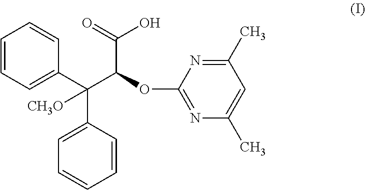

### FIELD OF THE INVENTION

The present invention relates to an improved and novel process for the preparation of highly pure (>99.8%) (+)-2(S)-(4,6-Dimethylpyrimidin-2-yloxy)-3-methoxy-3,3-diphenylpropionic acid (Ambrisentan) of formula-I

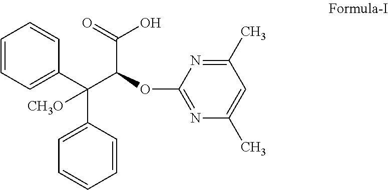

### BACKGROUND OF THE INVENTION

Ambrisentan which is (+)-2(S)-(4,6-Dimethylpyrimidin-2-yloxy)-3-methoxy-3,3-diphenylpropionic acid having the formula-I is approved under the trademark “Letairis” by the US Food and Drug Administration for the treatment of Pulmonary artery hypertension (PAH).

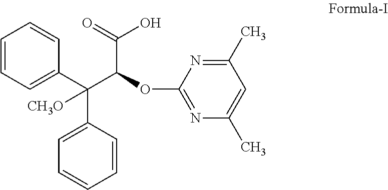

The preparation of (+)-2(S)-(4,6-Dimethylpyrimidin-2-yloxy)-3-methoxy-3,3-diphenylpropionic acid (Ambrisentan) having the formula-I is described in WO 9611914; U.S. Pat. No. 5,932,730(1996, 1998 both to BASF) and J. Med. Chem., 1996, vol. 39, No. 11, p.no. 2123-2128

In WO 9611914 and in its equivalent U.S. Pat. No. 5,932,730 the following route is described (Scheme-1) for related molecules. The route shown below is adapted for ambrisentan for our study.

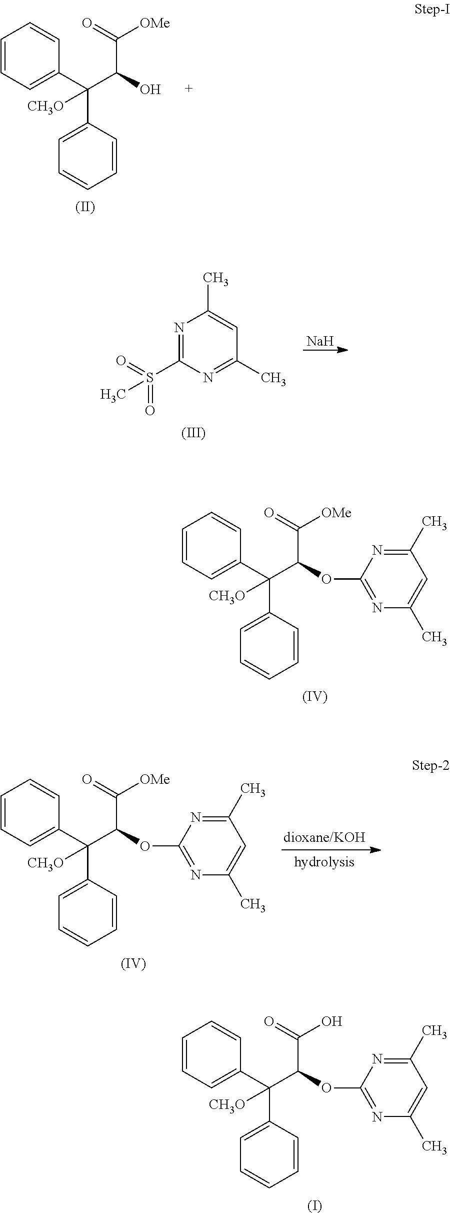

In this process methyl 2-hydroxy-3-methoxy-3,3-diphenylpropionate (II) is dissolved in DMF and sodium hydride is added. The mixture is stirred for a hour and then 4,6-dimethyl-2-(methylsulfonyl)pyrimidine is added. After stirring at room temperature for 24 hours cautious hydrolysis is carried out with water, the pH is adjusted to 5 with acetic acid., and the solvent is removed under high vacuum. The residue is taken up in ethyl acetate, washed with water and the solvent is distilled out. The residue is mixed with ether and the resulting precipitate is filtered off.

In step-2 the step-1 product is hydrolyzed in 1N KOH solution in dioxane medium at reflux temperature. After reaction completion the reaction mass is washed with ethyl acetate to remove unreacted ester. The pH of the aqueous layer is adjusted with concd. HCl pH 1-2 and extracted with ethyl acetate. After water washing, ethyl acetate is distilled off and the product was liberated by the addition of ether/hexane mixture.
The above process adds two more steps to the Route of synthesis viz., esterification and hydrolysis
1. racemic 2-hydroxy-3-methoxy-3,3-diphenylpropionic acid resolution followed by esterification
2. Condensation of the ester with 4,6-dimethyl-2-(methylsulfonyl)pyrimidine to yield Ambrisentan ester followed by hydrolysis to give Ambrisentan
3. When this process is repeated in our laboratory the overall realized yield of final product is less than 15%
4. The purity of the final product obtained is only 95%

In J. Med. Chem., 1996, vol. 39, No. 11, p.no. 2123-2128 same chemical route is described using potassium carbonate base in place of sodium hydride at 90° C. for step-1.
The overall realized yield of final product is less than 10% with a purity of about 92%

Further the preparation of (+)-2(S)-(4,6-Dimethylpyrimidin-2-yloxy)-3-methoxy-3,3-diphenylpropionic acid (Ambrisentan) having the formula-I is described in WO 01/05771(2001 to BASF)

In WO 9611914 the following route is described (Scheme-2) for molecules structurally related to ambrisentan.

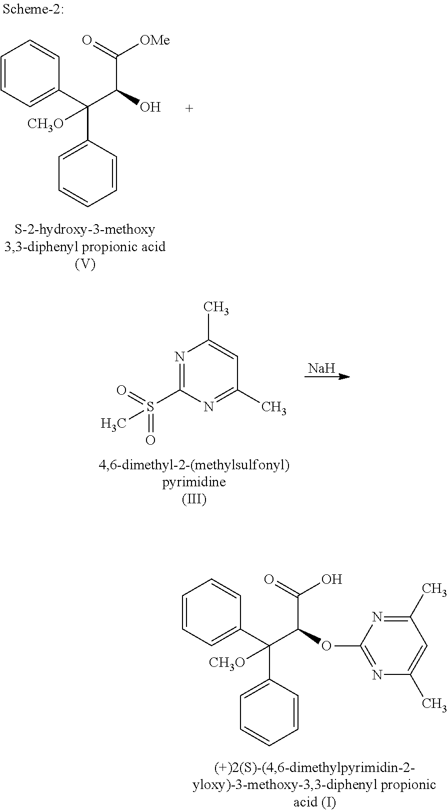

In this process S-2-hydroxy-3-methoxy-3,3-diphenylpropionic acid (V) is dissolved in DMF is added dropwise to a suspension of sodium hydride in DMF. The mixture is stirred for an hour and then 4,6-dimethyl-2-methylsulfonylpyrimidine in DMF is added. After stirring at room temperature for 24 hours it is poured into ice-water, the pH was adjusted to 1 with 2N HCl, and extracted with diethyl ether. The ether phase is extracted with 1N KOH, and the alkaline aqueous phase is readjusted to pH 1 with 2N HCl and extracted with ether. The solvent is stripped off in vacuum. The residue is stirred in diethyl ether overnight, filtered and dried. The solid obtained in this way is chromatographed on silica gel, allowing isolation of the desired product.

In this process mole ratio of S-2-hydroxy-3-methoxy-3,3-diphenylpropionic acid, 4,6-dimethyl-2-methylsulfonylpyrimidine and sodium hydride is 1:1.3:3.0.

By following this method in our laboratory, our observations are:
the reaction is not going to completion.
Chemical purity of only 95% is realized
chiral purity of the product is less than 95%
column chromatography is necessary to isolate the pure product

There is therefore an unfulfilled need to provide an industrially feasible process for the preparation Ambrisentan devoid of above shortcomings.

The objective of this invention is to prepare highly pure Ambrisentan through acid addition salts (1:1) of Ambrisentan. When the base is liberated from the acid addition salts, Ambrisentan of higher purity results.

It is surprisingly found by the inventors that when the less pure Ambrisentan is reacted with

S(−)4-nitro phenyl ethylamine or S(−)-phenyl ethyl amine it selectively forms the corresponding acid addition salt, leaving behind the other related substances and impurities which are otherwise difficult to remove by the conventional methods. The S(−) 4-nitrophenyl ethyl amine or S(−)-phenyl ethyl amine salt of Ambrisentan is further converted to highly pure Ambrisentan which in turn is converted into other pharmaceutically acceptable salts with higher purity.

### SUMMARY OF THE INVENTION

The main object of the present invention is to provide an improved process for the preparation of highly pure (>99.8%) Ambrisentan

Another object of the invention is to provide a process for preparation of salts of Ambrisentan with S(−)4-nitro phenyl ethyl amine or S(−)-phenyl ethyl amine in high purity (>99.8%).

Accordingly in the present invention highly pure Ambrisentan is prepared by

i. Preparing Ambrisentan by the condensation of S(+)2-Hydroxy-3-methoxy-3,3-diphenylpropionic acid with 2-(methylsulfonyl)-4,6-dimethylpyrimidine in presence of sodium hydride base in polar aprotic solvents like DMF or THF
ii. Treating Ambrisentan with S(−)4-nitro phenyl ethylamine or S(−)-phenyl ethyl amine yielding the corresponding addition salt of Ambrisentan
iii. Acidifying Ambrisentan S(−)4-nitro phenyl ethylamine or S(−)-phenyl ethyl amine salt and isolating Ambrisentan of purity 99.9%

### DETAILED DESCRIPTION OF THE INVENTION

Thus in accordance with the present invention preparation of Ambrisentan comprises of the following steps

i. preparing Ambrisentan by the condensation of S(+)2—Hydroxy-3-methoxy-3,3-diphenylpropionic acid with 2-(methylsulfonyl)-4,6-dimethylpyrimidine in presence of sodium hydride base in DMF/THF medium in 1:1.4:4.3 mole ratio
ii. Treating Ambrisentan with S(−)4-nitro phenyl ethylamine or S(−)-phenyl ethyl amine yielding corresponding addition salts of Ambrisentan
iii. Acidifying Ambrisentan S(−)4-nitrophenylethylamine or S(−)-phenyl ethyl amine salt and isolating Ambrisentan of purity 99.85%

In a typical embodiment, the present invention provides the following process for the preparation of Ambrisentan
1. S(+) 2-hydroxy-3-methoxy-3,3-diphenylpropionic acid (HIP-III) is dissolved in DMF/THF under nitrogen atmosphere at 20-25° C.
2. Sodium hydride is added slowly to the reaction mass during 1 hour at 25-30° C.
3, The mixture is stirred for a hour and then 4,6-dimethyl-2-(methylsulfonyl)pyrimidine in DMF/THF is added drop wise
4. The reaction mass is maintained at 25-30° C. for 16-17 hours
5. After maintenance it is quenched with methanol and poured into ice-water.
6. aqueous layer pH is adjusted with 1N hydrochloride solution to 2-3 during 30-45 minutes
7. Reaction mass is extracted with Ethyl acetate
8. Ethyl acetate layer is extracted with diluted sodium hydroxide solution
9. Sodium hydroxide layer is acidified with diluted hydrochloric acid
10. reaction mass is maintained under stirring for 2 hours at RT
11. The product is Filtered and dried to yield Ambrisentan

Further reacting the resultant base of Ambrisentan with S-(−)4-nitro phenylethylamine or S(−)-phenyl ethyl amine as follows:

i. Ambrisentan is dissolved in acetone and S-(−)4-nitro phenylethylamine/S-(−)-phenyl ethyl amine is added directly or as a solution in acetone
ii. reaction mass temperature is raised to reflux
iii. reaction mass is maintained at reflux temperature for 1 hours
iv. reaction mass is brought to room temperature and maintained at the same temperature for
16-18 hours
   iv. The product after filtration and drying at 60-70° C. afforded pure Ambrisentan as corresponding acid addition salt of S-(−)4-nitro phenyl ethylamine or S(−)-phenyl ethylamine

The prepared Ambrisentan S-(−)4-nitro phenyl ethyl amine/S(−)-phenyl ethyl amine acid addition salts (1:1) are novel and are identified and characterized by chemical, analysis, IR, NMR & Mass spectral. Ambrisentan acid addition salts are further converted to Ambrisentan as follows
i. Ambrisentan S-(−)4-nitro phenyl ethyl amine or S(−)-phenyl ethyl amine addition salt is acidified with diluted hydrochloric acid
ii. The reaction mass is maintained at room temperature for 2-3 hours
iii. The product is filtered and washed with purified water

The solid state properties of Ambrisentan thus prepared are illustrated by the following figures:

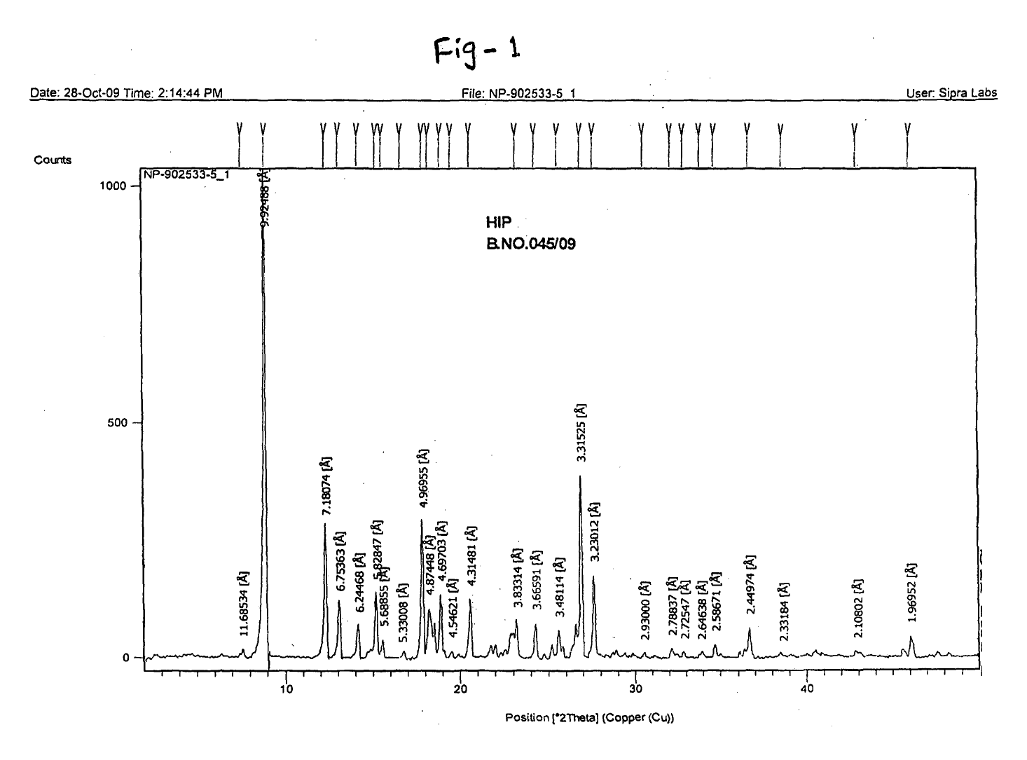

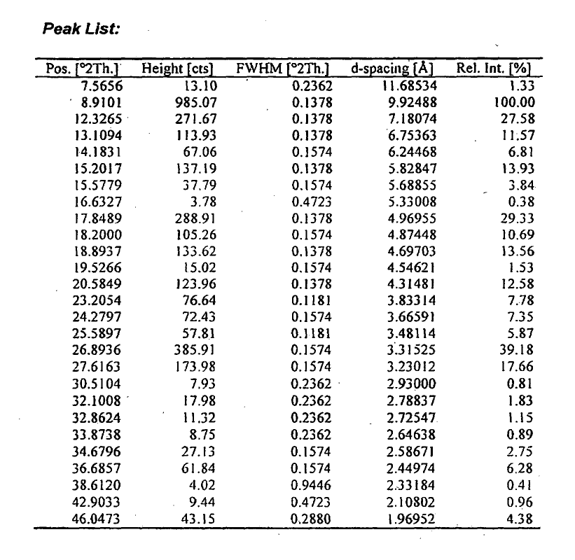

FIG. 1—XRPD spectrum of the Ambrisentan prepared by the method disclosed in example-1

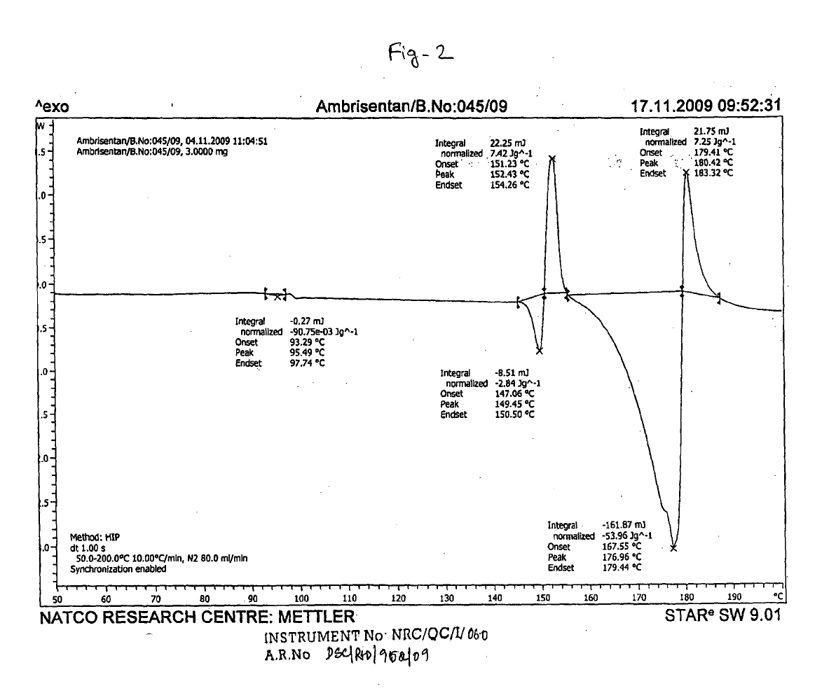

FIG. 2—DSC curve of the Ambrisentan prepared by the method disclosed in example-1

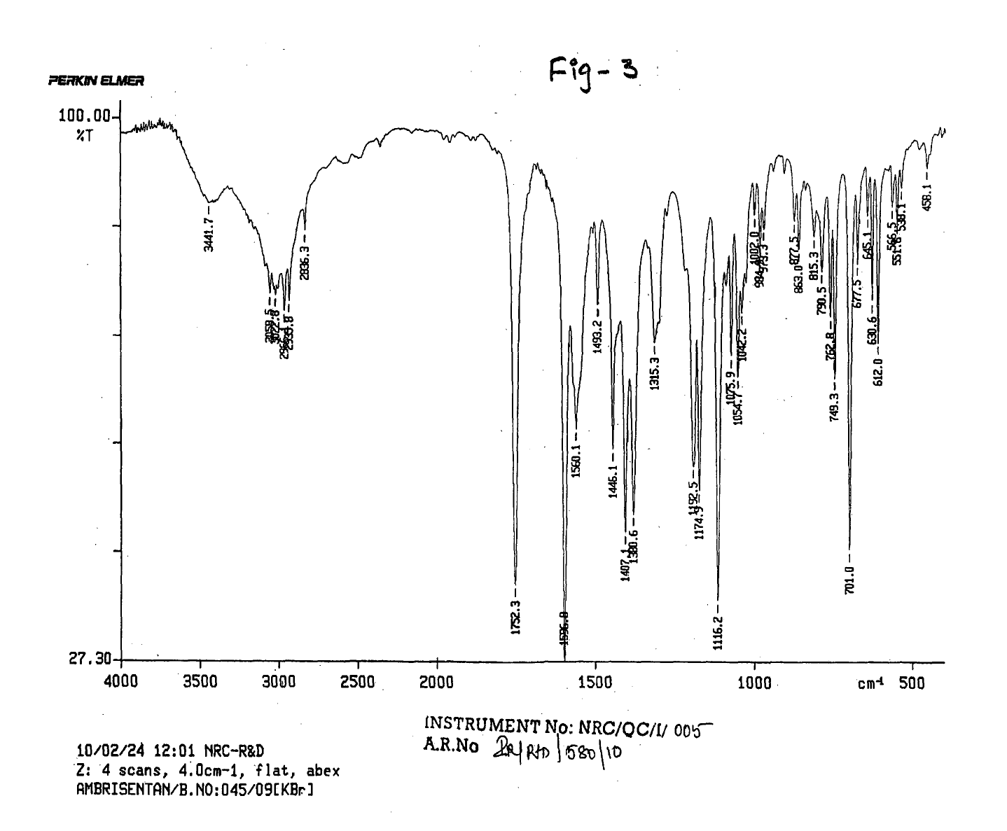

FIG. 3—IR spectrum of the Ambrisentan prepared by the method disclosed in example-1

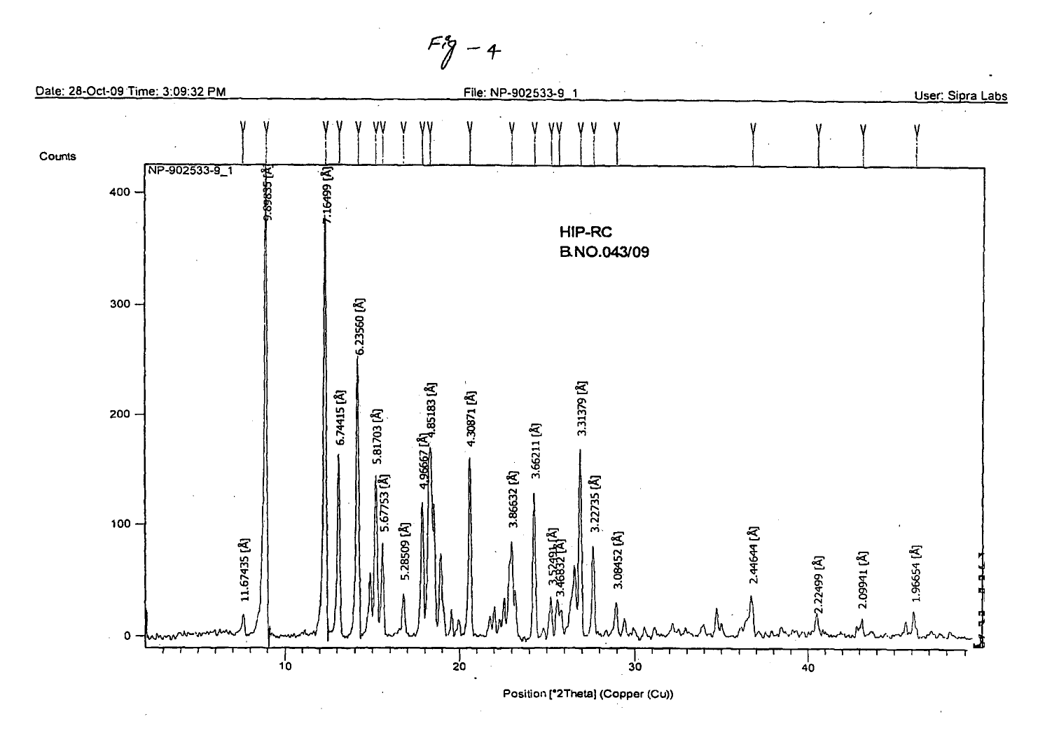

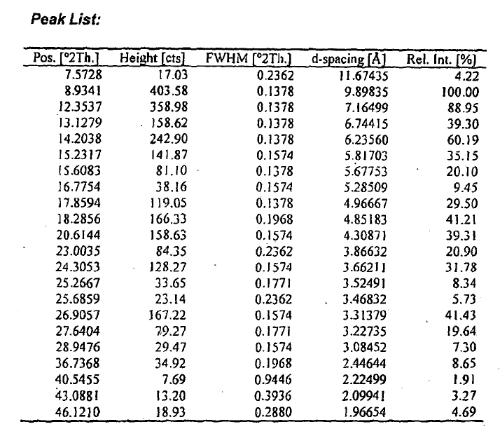

FIG. 4—XRPD spectrum of the Ambrisentan prepared by the method disclosed in example-2

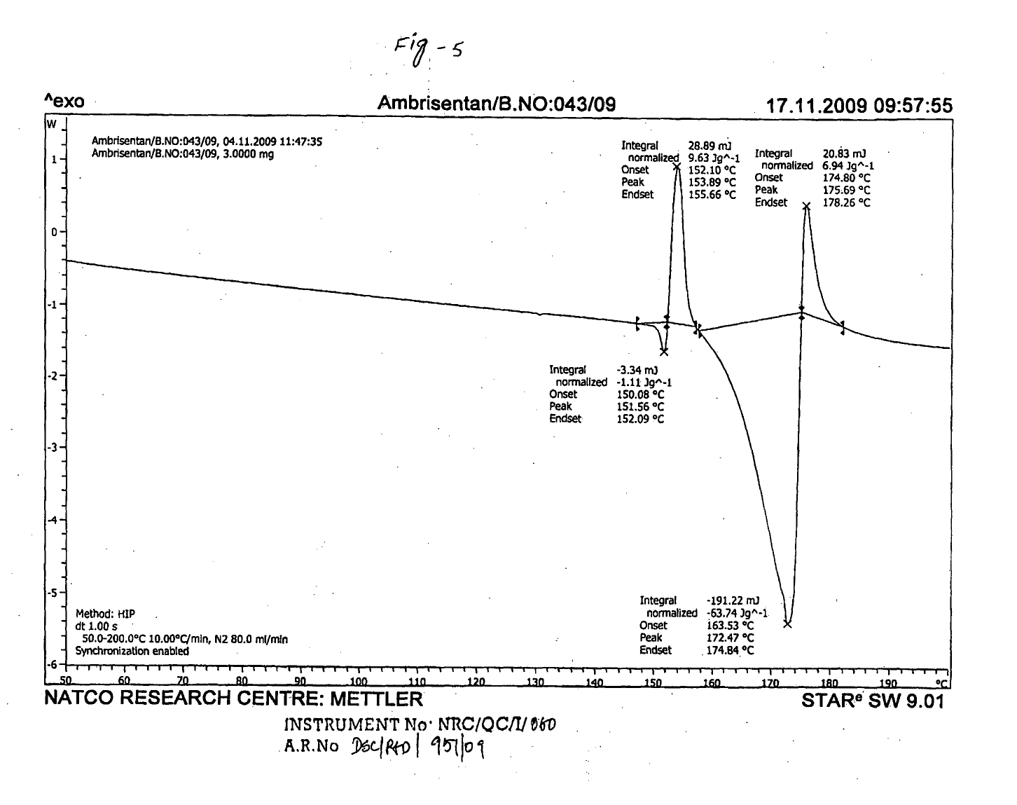

FIG. 5—DSC curve of the Ambrisentan prepared by the method disclosed in example-2

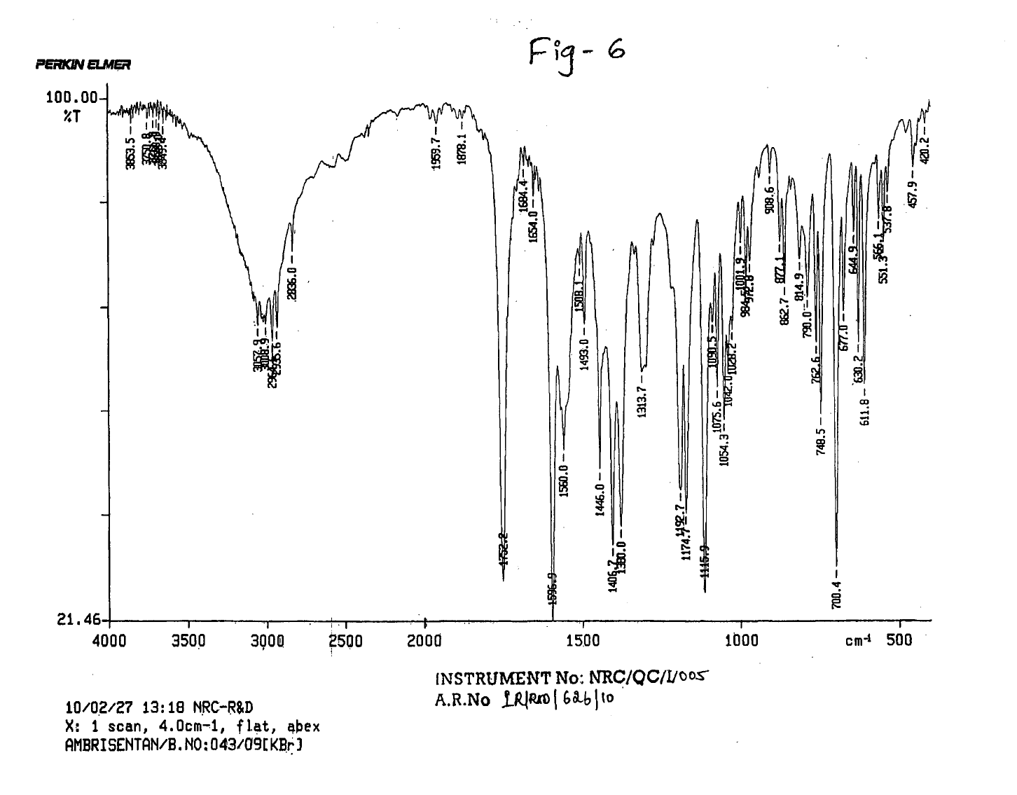

FIG. 6—IR spectrum of the Ambrisentan prepared by the method disclosed in example-2

The required S-2-hydroxy-3-methoxy-3,3-diphenylpropionic acid and 4,6-dimethyl-2-(methylsulfonyl)pyrimidine can be prepared by the prior art processes

The details of the inventions are given in the Examples which are provided for illustration only and therefore the Examples should not be construed to limit the scope of the invention.

### EXAMPLES

### Example-1

### Process for the Preparation Highly Pure Ambrisentan of the Formula-I

Step-1: Condensation of S(+)2-hydroxy-3-methoxy-3,3-diphenylpropionic acid and 4,6-dimethyl-2-(-(methylsulfonyl)pyrimidine in DMF medium

Into a 1 L round bottomed flask a mixture of DMF (400 ml) and S-2-Hydroxy-3-mehoxy-3,3-diphenyl propionic acid (50 g) were, charged and stirred for 30 minutes. sodium hydride (18.9 g) was added slowly for 1 hour and reaction mass was maintained at room temperature for one hours. 2-(methylsulfonyl)-4,6-dimethylpyrimidine (47.8 g) was dissolved in DMF (100 ml) and added to the reaction mass at room temperature during 45-60 minutes and reaction mass was maintained overnight under stirring. After reaction completion methanol (50 ml)1 was added slowly to the reaction mass during 30 minutes. Reaction mass was quenched into DM water (5 L) and acidified with diluted hydrochloric acid (600 ml). Aqueous layer was extracted with ethyl acetate (2×500 ml) and combined ethyl acetate layer was extracted with 1N sodium hydroxide solution. Sodium hydroxide layer was separated and acidified with 1N hydrochloride solution. Reaction mass was maintained under stirring for 2 hours. The product of the formula-I was filtered and washed with purified water. It was dried in oven at 60-65° C.

Dry weight: 60 g
Purity by HPLC: related: 99.5%
Chiral: 99.5%
   Step-II: Preparation of Ambrisentan S(−)4-nitro phenyl ethylamine addition salt (1:1): Ambrisentan (60 g, purity 99.5%) was dissolved in acetone (900 ml) and S-(−)4-nitro phenyl ethyl amine (26.2 g) was added to the solution over 30 min. Reaction mass' temperature was raised to reflux and maintained for about 1-2 hrs. Reaction mass was slowly cooled to room temperature and maintained for about 16-18 hr at the same temperature. The precipitated material was filtered and washed with 200 ml of acetone. The product was dried at 60-70° C. under vacuum till constant weight.
   Dry weight: 60 g
   Melting point: 156-160-deg C
   Purity by HPLC: related: 99.95% (Single impurity less than 0.1%)
Chiral: 99.85% (single impurity less than 0.1%)
   Step-III: Preparation of highly pure Ambrisentan from Ambrisentan S(−)P-nitro phenyl ethylamine addition salt (1:1):

Ambrisentan .S(−)4-nitro phenyl ethyl amine addition salt (60 g) was suspended in DM water (3 L) and stirred for 15 minutes. Aqueous 1N hydrochloric acid solution (500) was added over a period of 30 min to a pH of 1-2 and maintained at the same temperature for 2-3 hours. The precipitated product was filtered and washed with purified water. The product was dried at temperature of 60-70° C. till constant weight.

Dry weight of Ambrisentan: 42 g
Purity by HPLC: related: 99.95% (Single impurity less than 0.1%)
Chiral purity: 99.85% (Single impurity less than 0.1%)

### Example-2

### Process for the Preparation Highly Pure Ambrisentan of the Formula-I

Step-1: Condensation of S(+)2-hydroxy-3-methoxy-3,3-diphenylpropionic acid and 4,6-dimethyl-2-(-(methylsulfonyl)pyrimidine in THF medium

Into a 1 L round bottomed flask a mixture of THF (1 L) and S-2-Hydroxy-3-mehoxy-3,3-diphenyl propionic acid (50 g) were charged and stirred for 30 minutes. sodium hydride (18.9 g) was added slowly for 1 hour and reaction mass was maintained at room temperature for one hours. 2-(methylsulfonyl)-4,6-dimethylpyrimidine (47.8 g) was dissolved in THF (500 ml) and added to the reaction mass at room temperature during 45-60 minutes and reaction mass was maintained overnight under stirring. After reaction completion methanol (50 ml) was added slowly to the reaction mass during 30 minutes. Reaction mass was quenched into DM water (15 L) and acidified with diluted hydrochloric acid (600 ml). Reaction mass was maintained under stirring for 3 hours at room temperature. Filtered compound was dissolved in Ethyl acetate and ethyl acetate layer was extracted with 1N sodium hydroxide solution (2 L) Sodium hydroxide layer was separated and acidified with 1N hydrochloride solution (1.25 L). Reaction mass was maintained under stirring for 2 hours. The product of the formula-I was filtered and washed with purified water. It was dried in oven at 60-65° C.

Dry weight: 50 g
Purity by HPLC: related: 99.4%
Chiral: 99.36%
   Step-II: Preparation of Ambrisentan S(−) phenyl ethylamine addition salt (1:1): Ambrisentan (50 g, purity 99.5%) was dissolved in acetone (500 ml) and S-(−) phenyl ethyl amine (16.0 g) was dissolved in acetone (32 ml) added to the solution over 30 min. Reaction mass temperature was raised to reflux and maintained for about 1-2 hrs. Reaction mass was slowly cooled to room temperature and maintained for about 16-18 hr at the same temperature. The precipitated material was filtered and washed with 200 ml of acetone. The product was dried at 60-70° C. under vacuum till constant weight.
   Dry weight: 40 g
   Melting point: 88-90-deg C
   Purity by HPLC: related: 99.90% (Single impurity less than 0.1%)
Chiral: 99.82% (Single impurity less than 0.1%)
   Step-III: Preparation of highly pure Ambrisentan from Ambrisentan S(−) phenyl ethylamine addition salt (1:1):
   Ambrisentan .S(−) phenyl ethyl amine addition salt (40 g) was suspended in DM water (2 L) and stirred for 15 minutes. Aqueous 1N hydrochloric acid solution (330 ml) was added over a period of 30 min to a pH of 1-2 and maintained at the same temperature for 2-3 hours. The precipitated product was filtered and washed with purified water. The product was dried at temperature of 60-70° C. till constant weight.
   Dry weight of Ambrisentan: 30 g
   Purity by HPLC: related: 99.90% (Single impurity less than 0.1%)
Chiral purity: 99.80% (Single impurity less than 0.1%)

### ADVANTAGES OF THE INVENTION

1) Ambrisentan produced in more than 99.8% chemical purity.
2) The chiral purity of Ambrisentan by the process of the present invention is about 99.8%

1. Novel process for the preparation of Ambrisentan comprising
a) Dissolving S(+)2-hydroxy-3-methoxy-3,3-diphenylpropionic acid in DMF under nitrogen atmosphere at 20-25° C.
b) Adding Sodium hydride slowly to the reaction mass during 1 hour at 25-30° C.
c) stirring reaction mixture for one hour
d) adding 4,6-dimethyl-2-(methylsulfonyl)pyrimidine solution in DMF drop wise
e) Maintaining reaction mass at 25-30° C. for 16-17 hours
f) quenching reaction mass with methanol and pouring into ice-water.
g) aqueous layer pH adjustment with hydrochloride solution
h) extraction of reaction mass with ethyl acetate
i) extraction of ethyl acetate layer with sodium hydroxide solution
j) Acidification of separated sodium hydroxide layer with hydrochloric acid solution
k) Maintenance of reaction mass under stirring for 2 hours
l) Filtering to yield Ambrisentan.

2. Novel process for the preparation of highly pure (>99.8%) Ambrisentan comprising the following steps
I. Dissolving Ambrisentan in acetone and addition of S-(−)4-nitro phenyl ethyl amine directly or as a solution in acetone
II. Raising reaction mass temperature to reflux
III. Maintenance of reaction mass at reflux temperature for 1 hour
IV. Cooling reaction mass to room temperature and maintaining at the same temperature for 16-18 hours
V. Filtering and to yield pure Ambrisentan as an acid addition salt of S-(−)4-nitro phenylethylamine
VI. Acidification of Ambrisentan S-(−)4-nitro phenyl ethyl amine with diluted hydrochloric acid
VII. Maintenance at room temperature for 2-3 hours
VIII. Filtering to yield Ambrisentan of high purity (>99.8%)

3. Novel process for the preparation of Ambrisentan comprising
a) Dissolving S(+)2-hydroxy-3-methoxy-3,3-diphenylpropionic acid in THF under nitrogen atmosphere at 20-25° C.
b) Adding Sodium hydride slowly to the reaction mass during 1 hour at 25-30° C.
c) stirring reaction mixture for one hour
d) adding 4,6-dimethyl-2-(methylsulfonyl)pyrimidine solution in THF drop wise
e) Maintaining reaction mass at 25-30° C. for 16-17 hours
f) quenching reaction mass with methanol and pouring into ice-water.
g) aqueous layer pH adjustment with hydrochloride solution
h) Maintaining reaction mass under stirring for 3 hours
i) Filtration and dissolution of filtered solid in ethyl acetate
j) extraction of ethyl acetate layer with sodium hydroxide solution
k) Acidification of separated sodium hydroxide layer with hydrochloric acid solution
l) Maintenance of reaction mass under stirring for 2 hours
m) Filtering to yield Ambrisentan

4. Novel process for the preparation of highly pure (>99.8) Ambrisentan comprising the following steps
I. Dissolving Ambrisentan in acetone and addition of S-(−) phenyl ethyl amine directly or as a solution in acetone
II. Raising reaction mass temperature to reflux
III. Maintenance of reaction mass at reflux temperature for 1 hour
IV. Cooling reaction mass to room temperature and maintaining at the same temperature for 16-18 hours
V. Filtering and to yield pure Ambrisentan as an acid addition salt of S-(−) phenyl ethylamine
VI. Acidification of Ambrisentan S-(−) phenyl ethyl amine addition salt with diluted hydrochloric acid
VII. Maintenance at room temperature for 2-3 hours
VIII. Filtering to yield Ambrisentan of high purity (>99.8%)

5. A (1:1) addition salt of ambrisentan and S-(−)4-nitro phenyl ethyl amine as a novel pharmaceutically acceptable salt of Ambrisentan

6. A (1:1) addition salt of ambrisentan and S-(−) phenyl ethyl amine as a novel pharmaceutically acceptable salt of Ambrisentan

7. (canceled)

8. A process according to claim 1 wherein the hydrochloride solution in step “g)” is a 1N hydrochloride solution, the sodium hydroxide solution in step “i)” is a 1N sodium hydroxide solution, and/or the hydrochloric acid solution in step “j)” is a 1N hydrochloric acid solution.

9. A process according to claim 4 wherein the hydrochloride solution in step “g)” is a 1N hydrochloride solution, the sodium hydroxide solution in step “j)” is a 1N sodium hydroxide solution, and/or the hydrochloric acid solution in step “k)” is a 1N hydrochloric acid solution.
